(19) United States
(12) Patent Application Publication
(10) Pub. No.: US 2024/0382143 A1
(43) Pub. Date: Nov. 21, 2024

(54) COGNITIVE MODEL-BASED COGNITIVE STATE EVALUTION SYSTEM BASED ON COGNITIVE MODEL FOR SUBSTITUTING COGNITIVE TEST TASK USING LEARNING-BASED USER-CUSTOMIZED COGNITIVE MODEL

(71) Applicant: EMOTIV CO., LTD., Seoul (KR)

(72) Inventors: Jung Sang MIN, Seoul (KR); Ga Young KIM, Seoul (KR); Sung Jin KIM, Seoul (KR); Min young JUNG, Seoul (KR); Min Jung JI, Seoul (KR)

(21) Appl. No.: 18/691,855

(22) PCT Filed: Sep. 27, 2022
(86) PCT No.: PCT/KR2022/014483

§ 371 Date: Mar. 13, 2024

(30) Foreign Application Priority Data
Sep. 27, 2021 (KR) 10-2021-0126845

(51) Int. Cl. A61B 5/00; G16H 50/20; G16H 50/50

(52) U.S. Cl. CPC A61B 5/4088; G16H 50/20; G16H 50/50

(57) ABSTRACT

A method of operating a cognitive state evaluation apparatus according to an embodiment of the present invention comprises the steps of extracting first game data for cognitive evaluation from a user's response input information to a cognitive game application; creating a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and obtaining a substitution performance result of the cognitive task selected for each cognitive evaluation item using the customized cognitive task performance model and evaluating the cognitive ability of the user for each cognitive item based on the substitution performance result.

### TECHNICAL FIELD

The present invention relates to a cognitive state evaluation apparatus and an operation method thereof. More specifically, the present invention relates to a cognitive state evaluation system based on a cognitive model for substituting a cognitive test task using a learning-based user-customized cognitive model.

### BACKGROUND ART

Parents' deep knowledge regarding cognitive development status strengthens their ability to respond appropriately to their children, and thus providing the parents with information about their children makes it more easy to solve problems, gives confidence in their decisions, and enables a more sensitive response to the necessary requirements for children as they grow.

Accordingly, parents visit specialized diagnostic institutions with their children to determine the developmental status of their children, and their children receive medical tests, standardized tests (e.g., social maturity test, KEDI-WISC, portage cognitive development test, etc.), informal tests, observation, filling-out questionnaire, interview, etc.

In addition, elderly people with dementia or people who are expected to suffer from depression, etc. visit specialized diagnostic institutions to receive various cognitive tests.

However, conventional diagnostic methods take at least 2 hours or more as they perform numerous medical examinations and tasks, and diagnosis subjects such as children and the elderly may refuse to get diagnosed themselves or feel tired during diagnosis thereby causing problems that the medical examinations or tasks cannot be normally performed.

The present invention has been created to solve the above problems, and its purpose is to provide a cognitive state evaluation system wherein a learning-based user-customized cognitive model for diagnosing a cognitive state is built-up by extracting game data from a user terminal's input information for a cognitive game provided for a short period of time, and based on the user-customized cognitive model, tasks necessary for diagnosing cognitive states are processed so that they are automatically performed in the user-customized cognitive model, thereby dramatically reducing the time and fatigue required for diagnosing cognitive states, and enabling various diagnoses and evaluations.

An apparatus according to an embodiment of the present invention for solving the above problems is a cognitive state evaluation apparatus. The apparatus comprises a game data processing unit for extracting first game data for cognitive evaluation from a user's response input information to a cognitive game application; a customized cognitive task performance model configuration unit for creating a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and a cognitive ability evaluation unit for obtaining a substitution performance result of the cognitive task selected for each cognitive evaluation item using the customized cognitive task performance model and evaluating the cognitive ability of the user for each cognitive item based on the substitution performance result.

Also, a method according to an embodiment of the present invention for solving the above problems is a method of operating a cognitive state evaluation apparatus. The method comprises the steps of: extracting first game data for cognitive evaluation from a user's response input information to a cognitive game application; creating a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and obtaining a substitution performance result of the cognitive task selected for each cognitive evaluation item using the customized cognitive task performance model and evaluating the cognitive ability of the user for each cognitive item based on the substitution performance result.

Also, the method according to the embodiment of the present invention for solving the above problems may be implemented by a computer readable recording medium and a computer program for executing the method on a computer.

According to an embodiment of the present invention, a learning-based user-customized cognitive model for diagnosing a cognitive state is configured by extracting game data from input information of a user terminal for a cognitive game provided for a short time, and based on the user-customized cognitive model Cognitive state evaluation apparatus and its operation that drastically reduce the time and fatigue required for cognitive state diagnosis and enable various diagnosis and evaluation by processing tasks necessary for cognitive state diagnosis to be automatically performed in a user-customized cognitive model method can be provided.

Accordingly, according to the embodiments of the present invention, it can be used for education, medical care, etc. through personalized diagnosis and evaluation based on cognitive modeling, and various medical examinations such as ADHD tests which previously took about 2 hours, can be performed in the form of simple and short game.

In addition, according to an embodiment of the present invention, self-diagnosis for cognitive status is also possible, so that the effect of cognitive health promotion, which can lead to cognitive health status monitoring and rapid visit to the hospital and treatment, can also be promoted.

### BRIEF DESCRIPTION OF THE INVENTION

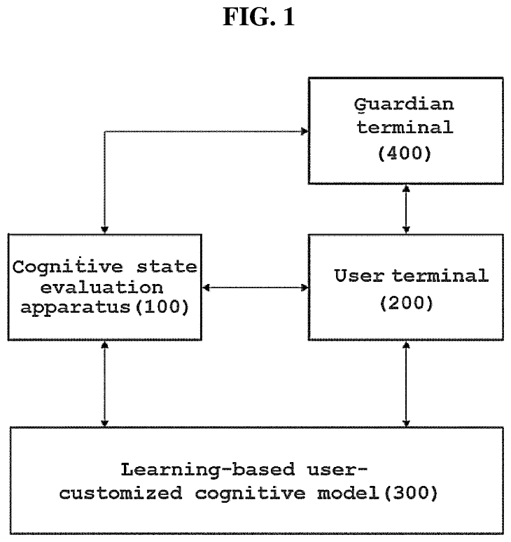

FIG. 1 is a conceptual diagram schematically showing the entire system of the present invention.

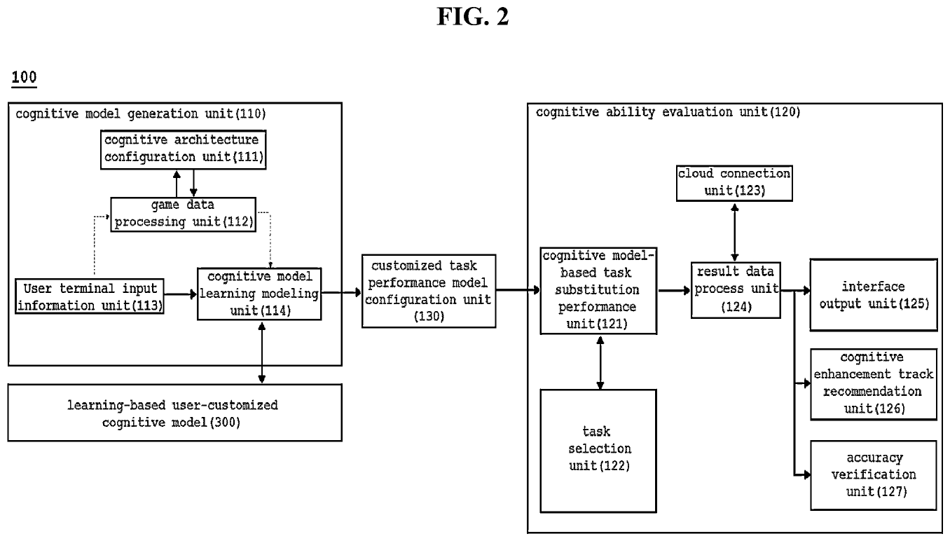

FIG. 2 is a block diagram illustrating in more detail a cognitive state evaluation apparatus according to an embodiment of the present invention.

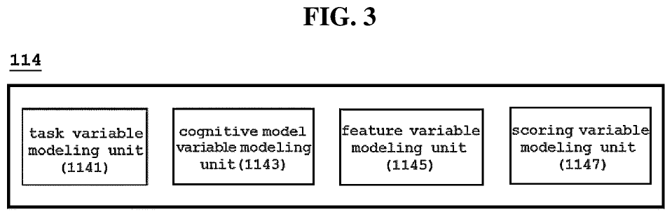

FIG. 3 is a diagram illustrating variable modeling data for configuring a cognitive architecture according to an embodiment of the present invention.

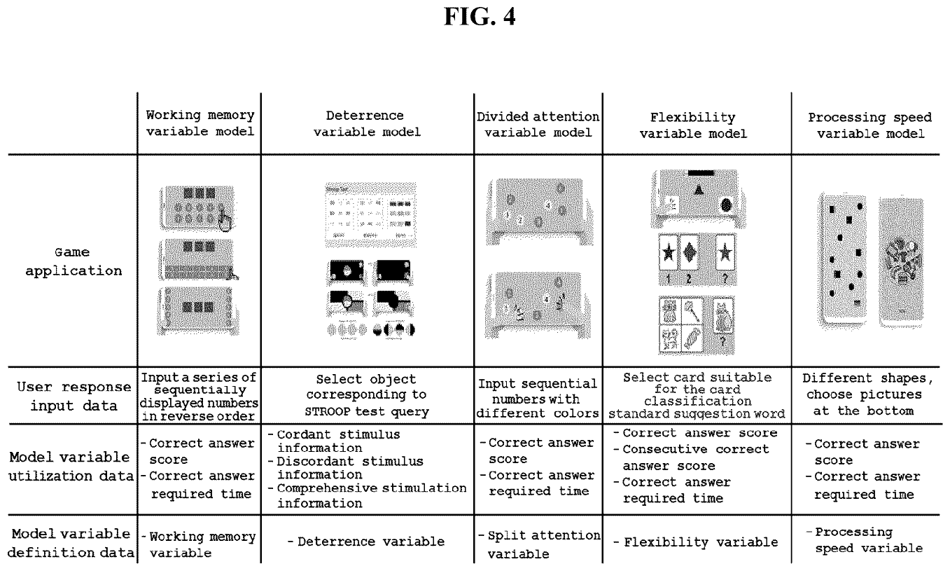

FIG. 4 is diagrams for explaining operations for each game application for calculating task model variables according to an embodiment of the present invention.

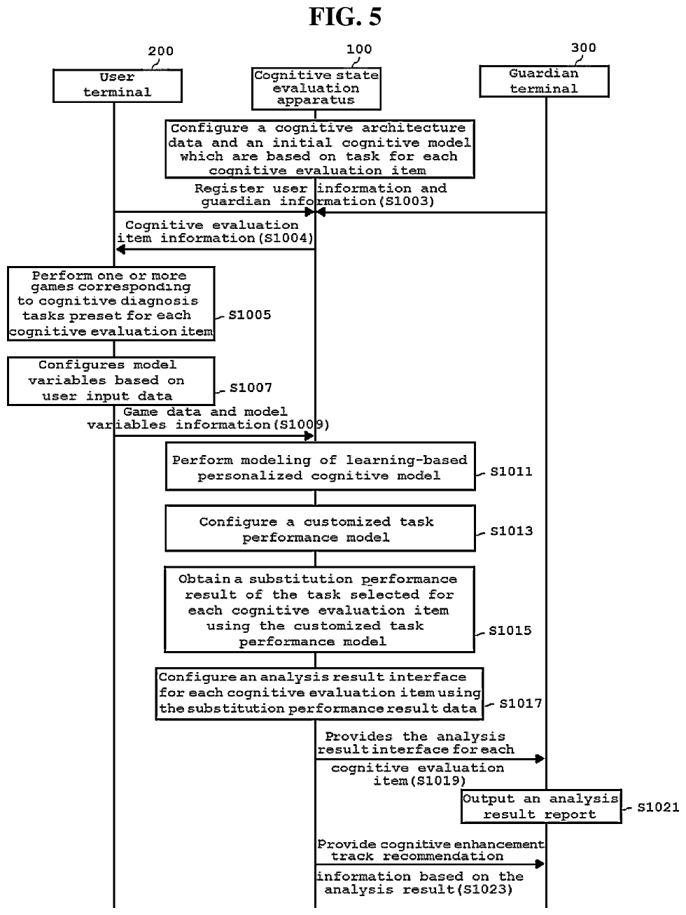

FIG. 5 is a ladder diagram for explaining in detail the entire system operation of the present invention.

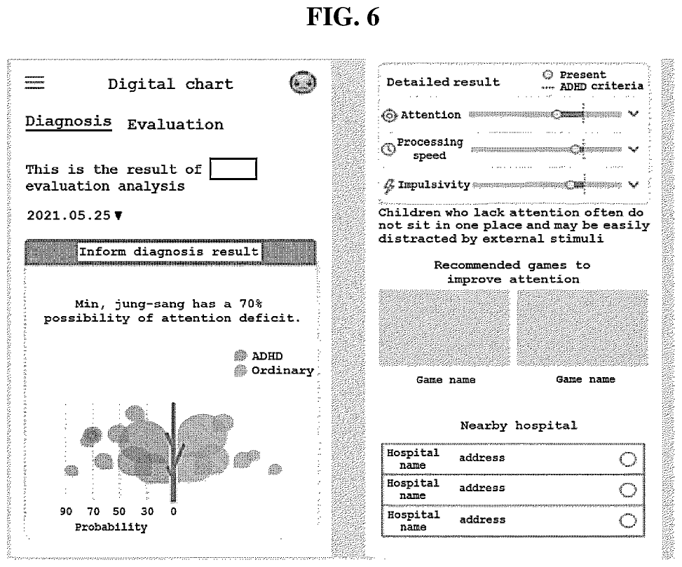

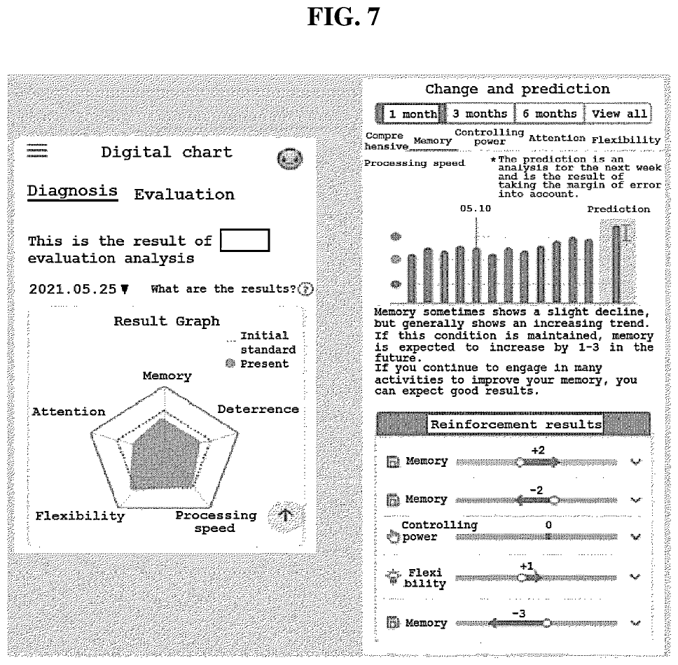

FIGS. 6 to 7 are diagrams for exemplarily explaining a report interface output from a guardian terminal according to an embodiment of the present invention.

### DETAILED DESCRIPTION OF THE INVENTION

The following merely illustrates the principles of the present invention. Therefore, those skilled in the art can invent various devices that embody the principles of the present invention and fall within the concept and scope of the present invention, even though not explicitly described or shown herein. In addition, it is to be understood that all conditional terms and embodiments listed herein are, in principle, expressly intended only for the purpose of making the concept of the present invention understood, and not limited to such specifically listed embodiments and conditions.

Further, it should be understood that all detailed descriptions reciting specific embodiments, as well as principles, aspects and embodiments of the present invention, are intended to encompass structural and functional equivalents of these matters.

In addition, it should be understood that such equivalents include not only currently known equivalents but also equivalents developed in the future, that is, all devices invented to perform the same function regardless of structure.

Thus, for example, the block diagrams herein are to be understood as representing conceptual views of exemplary circuits embodying the principles of the present invention. Similarly, all flowcharts, state transition diagrams, pseudo code, etc., are meant to be tangibly represented on computer readable media and represent various processes performed by a computer or processor, whether or not the computer or processor is explicitly depicted.

The functions of various elements shown in the drawings including functional blocks represented by processors or similar concepts may be provided using dedicated hardware as well as hardware capable of executing software in conjunction with appropriate software. When provided by a processor, the functionality may be provided by a single dedicated processor, a single shared processor, or a plurality of separate processors, and some of which may be shared.

In addition, the explicit use of terms presented as processor, control, or similar concepts should not be construed as exclusively referring to hardware capable of executing software, and it should be understood that they may include, without limitation, digital signal processor (DSP) hardware, ROM for storing software (ROM), random access memory (RAM) and non-volatile memory. Other well-known or commonly used hardware can be included.

In the claims of this specification, components expressed as means for performing the functions described in the detailed description are intended to encompass, for example, a combination of circuit elements performing the functions or all methods that performs the functions including all types of software including firmware/microcode, etc. and are combined with suitable circuitry for executing the software. Since the invention defined by these claims combines the functions provided by the various enumerated means and is combined in the manner required by the claims, any means capable of providing such functions is equivalent to that discerned from this disclosure. should be understood as

The above objects, features and advantages will become more apparent through the following detailed description in conjunction with the accompanying drawings, and accordingly, those skilled in the art to which the present invention belongs can easily implement the technical idea of the present invention. There will be. In addition, in describing the present invention, if it is determined that a detailed description of a known technology related to the present invention may unnecessarily obscure the subject matter of the present invention, the detailed description will be omitted.

Hereinafter, a preferred embodiment according to the present invention will be described in detail with reference to the accompanying drawings.

FIG. 1 is a conceptual diagram schematically illustrating an entire system according to an embodiment of the present invention.

The entire system according to an embodiment of the present invention includes a cognitive state evaluation apparatus 100, a user terminal 200, a guardian terminal 400, and a learning-based user-customized cognitive model 300.

In order to provide cognitive state diagnosis and evaluation services according to an embodiment of the present invention, the cognitive state evaluation apparatus 100 can be connected, through a wireless network, each of the user terminal 200, the guardian terminal 400 and the learning-based user-customized cognitive model 300 and perform inter-communications between them.

Here, each network can be implemented in all types of wired/wireless networks, such as a local area network (LAN), a wide area network (WAN), a value added network (VAN), a personal area network (PAN), a mobile radio communication network or a satellite communication network.

And, the user terminal 200 and the guardian terminal 400 may be an individual equipment of any one of a computer, a mobile phone, a smart phone, a smart pad, a laptop computer, a personal digital assistant (PDA) and a portable media player (PMP) or at least one multi-device among common-used devices such as a kiosk or a stationary display device installed in a specific place.

First, the user terminal 200 is a terminal pre-registered in the cognitive state evaluation apparatus 100 together with the guardian terminal 400, and may be a terminal apparatus of a cognitive evaluation subject. And, the user terminal 200 may output the cognitive game provided from the cognitive state evaluation apparatus 100 according to the cognitive evaluation items, receive user response data corresponding to the cognitive game and transmit the received user response data to the cognitive state evaluation apparatus 100.

In such a system configuration, the cognitive state evaluation apparatus 100 may pre-build up a learning-based user-customized cognitive model 300. The cognitive state evaluation apparatus 100 can compare cognitive game data extracted from user response data corresponding to a cognitive game with past cognitive state data diagnosed corresponding to learning subjects and can build up a learning-based user-customized cognitive model 300. The learning may for example, include various deep-learning methods such as CNN, DNN, RNN, LSTM, etc., and an analysis method such as regression analysis, etc. or a statistical relationship analysis method may also be used.

More specifically, can pre-build up a user-customized cognitive model based on artificial intelligence learning in which a cognitive architecture-based cognitive model has been pre-associatively learned in response to game data for each cognitive task.

Here, the cognitive architecture-based cognitive model can include a known adaptive control of thought rational (ACT-R) architecture-based cognitive model, and the cognitive evaluation items can include an attention deficit hyperactivity disorder (ADHD) evaluation item corresponding to the ACT-R, enabling evaluation of cognitive items corresponding to ADHD based on the game data.

To this end, the cognitive state evaluation apparatus 100 can first extracts a first game data for cognitive evaluation from response input information for the user's cognitive game application, apply the extracted first game data to the learning-based user-customized cognitive model 300, create customized cognitive task performance model corresponding to the user, obtain an substitution performance result of the cognitive task selected for each cognitive evaluation item using the customized cognitive task performance model and evaluate the user's cognitive ability for each of the cognitive items based on the substitution performance result.

Here, the task performance model is an automatic task performance model generated by predicting the user's cognitive state using a learning-based user-customized cognitive model 300 from game data input, and may be a model for a series of various questionnaires and tasks that take about two hours for diagnosing the cognitive state such as existing ADHD to be automatically substitution-performed without a separate user input.

That is, the task performance model is a model that outputs predicted result data when a user-customized cognitive model built with the game data has performed a task for cognitive evaluation, which can be predicted by predetermined evaluation criteria for each task. For example, when the response rate variable of the user-customized cognitive model is 0.5, the task performance model outputs the result predicted when the cognitive evaluation task is performed in the state where the response rate variable is 0.5, as the substitution performance result.

Accordingly, the cognitive game application according to an embodiment of the present invention may be configured to collect response variables for generating a task performance model. In addition, the cognitive state evaluation apparatus 100 may extract these response variables by type from the game data, and to do this, cognitive game applications configured step by step in advance may be provided to the user terminal 200.

These cognitive game applications may include cognitive games that can be performed in a short period of time, and may include cognitive games in which various tasks for extracting cognitive variables and the like used for diagnosing and evaluating cognitive states are sequentially or concurrently performed.

Meanwhile, the cognitive game application configured as described above may be outputted from the user terminal 200, and the subject person inputs a user response input to the user terminal 200 to perform the game. The user terminal 200 can process the input user response input information and transmit it to the cognitive state evaluation apparatus 100.

In addition, the cognitive state evaluation apparatus 100 generates a user-customized cognitive model by applying the cognitive game data extracted from the user response input information to the learning-based user-customized cognitive model 300, and uses the cognitive game data extracted from the user-customized cognitive model. A corresponding customized task performance model can be created.

Accordingly, the cognitive state evaluation apparatus 100 digitizes and inputs existing tasks configured for cognitive state evaluation by a person to the customized task performance model, thereby replacing the cognitive state of each user according to the alternative driving of the customized task performance model. Diagnosis result data may be obtained, and appropriate diagnosis and evaluation information based on the obtained result data may be quickly processed and provided to the user terminal 200 and the guardian terminal 400 or a separate institution.

FIG. 2 is a block diagram illustrating a cognitive state evaluation apparatus according to an embodiment of the present invention in more detail.

According to an embodiment of the present invention, the cognitive state evaluation apparatus 100 may include a cognitive model generator 110, a cognitive ability evaluation unit 120, and a customized task performance model configuration unit 130, and a learning-based user-customized cognitive model 300 may be connected to the cognitive state evaluation apparatus 100, included in the cognitive state evaluation apparatus 100, or pre-built in an external server or the like.

First, the cognitive model generation unit 110 includes a cognitive architecture configuration unit 111, a game data processing unit 112, a user terminal input information processing unit 113, and a cognitive model learning modeling unit 114.

First, the cognitive architecture configuration unit 111 stores and manages pre-configured architecture data of cognitive variables in order to extract variables for cognitive state evaluation and diagnosis from game data of a game application.

Then, the user's input information input to the user terminal input information processing unit 113 is processed as a cognitive variable in the game data processing unit 112 and applied to the cognitive model learning modeling unit 114.

Then, the cognitive model learning modeling unit 114 inputs the cognitive variables of the game data processing unit into the learning-based user-customized cognitive model 300 to build a customized task performance model.

More specifically, for example, the cognitive architecture configuration unit uses a known adaptive control of thought rational (ACT-R) architecture-based cognitive model to logically configure human cognition/behavioral processes, using preset modules and buffers. A cognitive model architecture based on condition-execution statements may be configured, and the game data processing unit 112 may perform a process of mapping each condition-execution statement of the cognitive model architecture to preset game data.

In addition, the cognitive model learning modeling unit can configure a personalized cognitive model tailored to the user by extracting cognitive model variables related to working memory, attention, cognitive flexibility, inhibition, processing speed, etc. which are obtained from resultant data of the user's performance of N games from the architecture mapping data of the game data processing unit 112 and the input information of the user terminal input information processing unit 113, and then applying the extracted cognitive model variables to the learning-based user-customized cognitive model (300).

In addition, the customized task performance model configuration unit 130 may configure a task-customized cognitive task performance model capable of substitutingly performing cognitive tasks selected for each cognitive evaluation item by using the personalized cognitive model.

To this end, the customized task performance model configuration unit 130 can configure the above-described ACT-R-based basic task performance model architecture wherein it can create a virtual task performance model configured to repeatedly measure a performance time, an error rate, a mission success rate, a correct answer rate, and a continuous success rate, etc. of each user when a task is inputted by applying variables for each personalized cognitive model.

The customized task performance model configuration unit 130 may individually generate this task performance model and further receive from the user terminal 200 game data after the N+lth that has not been used in modeling to verify the accuracy.

The game data after the N+lth may be transmitted to the accuracy verification unit 127 of the cognitive ability evaluation unit 120. The accuracy verification unit 127 may compare and detect an error in model prediction information in accordance with comparative evaluation based on performance time, error rate, latency, etc., and if the error is greater than or equal to a threshold value, determine to perform an additional game and provide a notification to the user terminal 200. Through such self-learning of the model, the customized task performance model configuration unit 130 may more accurately generate a user replication cognitive model for automatically substituting the customized task.

In addition, the cognitive ability evaluation unit 120 includes a cognitive model-based task substitution performance unit 121 for automatically performing a cognitive model-based task using a customized task performance model.

Here, the tasks may include diagnosis or questionnaire tasks that can be substitution-configured in accordance with each diagnosis purpose or subject, and the cognitive state evaluation apparatus 100 may further include a task selection unit 122 in which selectively configures the task according to each purpose of ADHD status evaluation and the like or user classification.

In addition, the cognitive state evaluation apparatus 100 includes a result data processing unit for processing result data, and the result data can be provided to the guardian terminal 400 or user terminal 200 through a cloud connection unit, outputted on the screen of the guardian terminal 400 through the interface output unit 125 or provided to the cognitive enhancement track recommendation unit 126 so that the cognitive enhancement process corresponding to the cognitive state diagnosis result may be provided to the guardian terminal 400 and processed to be outputted.

More specifically, the cognitive ability evaluation unit 120 can allow a cognitive model-based task substitution performance unit 121 to substitutingly perform various tasks, which the actual user has to perform, based on the substitution performance of the customized task performance model and to perform various tasks more than M times based on user-customized learned and replicated performance ability (how many missions have been performed, how many errors have occurred, which targets have been missed, etc.).

The result data processing unit 124 may perform quantification in accordance with a preset standard or a predefined algorithm to configure performance result data.

And, the interface output unit 125 may configure an analysis result interface using the quantified performance result data, and report data including the configured analysis result interface can be provided to the guardian terminal 400.

Accordingly, the guardian terminal 400 may provide the user's cognitive state evaluation information through various types of interfaces. For example, an analysis result interface may be reported in the form of a pentagonal spider map, and additionally analyzed information may be outputted as qualitative data.

Furthermore, the result data processing unit 124 may predict data for the next two weeks using statistical analysis based on information performed so far and provide the predicted data to the user terminal 200. To this end, the result data processing unit 124 can calculate a growth regression equation through the cognitive model and learning, and perform analysis processing for producing prediction data within two weeks.

In addition, the cognitive reinforcement track recommendation unit 126 can configure, if the preset cognitive state item is below the threshold, a reinforcement track, which is composed of reinforcement tasks capable of reinforcing the ability of the cognitive state item which is below the threshold, as a recommendation track and can perform a recommendation process for providing the configured recommendation track to the guardian terminal 400 or the user terminal 200.

Here, the recommended track information can be provided to the user terminal 200 or the guardian terminal 400, and the recommended track information may include one or more game information configured stepwisely. Here, each recommended game may include tasks that comprehensively improve cognitive ability, and in particular, tasks that more strongly and intensively reinforce the items that are below the above threshold predicted to be deficient.

For a series of such processes, all RAW data may be processed after primary storage in the user terminal 200 and provided to the cognitive state evaluation apparatus 100, and then the aggregated data may be stored in the cloud server through the cloud connection unit 123 and finally aggregated data can be provided to the user terminal 200 or the guardian terminal 400.

Through the establishment of such a system, personalized diagnosis/evaluation through cognitive modeling can be carried out quickly and easily, which can be used for education, medical (treatment), etc., and in particular, children's ADHD tests, which should have been typically took nearly two hours, can be processed more simply and quickly.

Also, 90% of potential children with ADHD in Korea do not go to the hospital wherein if the present cognitive state evaluation apparatus 100 is used, it has the effect capable of easily visiting the hospital after a simple self-diagnosis and the advantage of capable of being used not only for the children diagnosis but also for general cognitive diagnosis such as autism, depression, dementia, etc.

FIG. 3 is a diagram illustrating variable modeling data for configuring a cognitive architecture according to an embodiment of the present invention.

Referring to FIG. 3, the cognitive model learning modeling unit 114 includes a task variable modeling unit 1141 for allowing to perform each task based on the cognitive learning model, a cognitive model variable modeling unit 1143, and feature variable modeling unit 1145 and a scoring variable modeling unit 1147.

The task variable modeling unit 1141 can perform a process of modeling a user input result for each task extracted from each game data as a model variable for cognitive task performance for evaluating cognitive ability.

And, the cognitive model variable modeling unit 1143 can perform modeling for setting learning variables obtained according to the cognitive architecture in order to configure a cognitive model.

Here, the cognitive architecture can be exemplified as the ACT-R model. In the case of the ACT-R, it is configured that various roles of the human brain are shared by each of eight modules, and each module and a production system acting as a central processing unit are configured to exchange each other information (i.e., chunks) about the cognitive process through a buffer. At this time, although the buffer can sequentially store and process only one chunk at a time, each module can retrieve or store multiple pieces of information simultaneously, and in a production system can be configured such that tasks according to multiple production rules can be comparative-processed in parallel at the same time.

Accordingly, the cognitive model variable modeling unit 1143 may dataficate the buffer and system structure preset by the cognitive architecture, and select and adjust learning variables for configuring a personalized cognitive model.

And, the feature variable modeling unit 1145 may process modeling for extracting a feature variable useful for performing game data-based task substitution from variables modeled by the cognitive model variable modeling unit 1143. Here, the feature information may be a variable which is extracted through a known SVM method, etc. As the primary feature information, for example, an activation function for learning, a base level activation value, recognition accuracy, recognition delay time, etc. may be included, and a minimum value, a maximum value, an average, a standard deviation, and a variance of the primary feature information may be included as the secondary feature information.

Also, the scoring variable modeling unit 1147 can model an algorithm and scoring variables capable of calculating cognitive screening and cognitive evaluation scores corresponding to an individual wherein they may be processed by adjusting variables corresponding to evaluation function for each of one or more cognitive evaluation items which are calculated using previously set task variables, cognitive model variables and feature variables.

FIG. 4 is diagrams for explaining operation for each game application for calculating task model variables according to an embodiment of the present invention.

Referring to FIG. 4, task model variables calculated from game data according to an embodiment of the present invention can be largely exemplified by a working memory variable model, an deterrence variable model, a split attention variable model, a flexibility variable model, and a processing speed variable model. However, this is just an example and additional variable models may be further included.

First, the working memory variable model represents the ability to store information coming from other sensory organs in the head and retrieve the information again within a certain short time. The game application according thereto can sequentially indicate a series of numbers and the user can input response data to input a series of the sequentially indicated numbers in reverse order. And, correct answer scores, time required for correct answers and the like may be used as model variables, and as a result, the working memory variables may be reflected into a customized cognitive evaluation task performance model.

Also, the deterrence variable model represents a selective/intensive activity and state ability to clearly recognize only a specific one of many stimuli from the external environment or the inside of the object, or to respond only to the specific one, and the game application according thereto may include, as an example, a known stroop test and the like. The stroop test lists words or objects in which the display colors of the words or objects and the meaning of the words or objects are different from each other, and selects only a specific color or object to thereby check selective/intensive activity ability. A user can input response data to select an object corresponding to the test query. And, congruent stimulus information, incongruent stimulus information, synthetic stimulus information, etc. may be used as its model variables, and as a result, they may be reflected into a customized cognitive evaluation task performance model as deterrence variables.

And, the split attention variable model represents the ability to respond to various demands of the surrounding environment while responding to two different stimuli at the same time, and indicates whether two tasks can be performed simultaneously. The game application according thereto is an application that indicates sequential numbers having different colors, and the user can select sequential numbers having different colors and input them as response data. And, the model variables, such as the correct answer score and the time required for the correct answer may be used as its variables, and as a result, they may be reflected into the customized cognitive evaluation task performance model as the divided attention variables.

And, the flexibility variable model represents the mental ability to change thoughts and actions appropriately depending upon changes in the external environment and rules, and represents the ability to perform a change in thoughts to meet the required changes. The game application according thereto is an application that selects a card suitable for the card classification standard suggestion word, and the user can select an appropriate card according to the standard suggestion word and input it as response data. And, model variables such as correct answer score, continuous correct score, and time required for correct answer can be used as its variables, and as a result, they can be reflected into the customized cognitive evaluation task performance model as flexibility variables.

Also, the processing speed variable model represents the time to respond to a stimulus and the speed at which a user can understand and respond to whether the recognition information is visual, auditory, or kinematic. Accordingly, the game application is an application that selects a figure or object according to a query, and the user may select a figure of a different shape or a figure of a presented shape and input the result as response data.

Also, the processing speed variables model represents the time to respond to a stimulation and the speed at which can understand whether the recognition information is visual, auditory or athletic, etc. and respond thereto. The game application according thereto is an application that selects a figure or object depending upon a query wherein the user can select a figure of different shape or a figure of the presented shape and input it as response data. And, model variables such as correct answer score and time required for the correct answer can be used as its variables and as a result, they can be reflected into a customized cognitive evaluation task performance model as processing speed variables.

FIG. 5 is a ladder diagram for explaining the entire system operation of the present invention in detail.

Referring to FIG. 5, the cognitive state evaluation apparatus 100 according to an embodiment of the present invention first configures a cognitive architecture data and an initial cognitive model which are based on task for each cognitive evaluation item (S1001).

And, the cognitive state evaluation apparatus 100 registers user information of the user terminal 200 and guardian information of the guardian terminal 400 (S1003). Here, the user information may include, as an example, terminal identification information, user account information, phone number information, family relationship information and the like.

Thereafter, the user terminal 200 receives cognitive evaluation item information from the cognitive state evaluation apparatus 100 (S1004), and performs one or more games corresponding to cognitive diagnosis tasks preset for each cognitive evaluation item (S1005).

And, the user terminal 200 configures model variables based on user input data (S1007), and transmits the configured game data and model variables information to the cognitive state evaluation apparatus 100 (S1009). Here, the step S1007 may also be performed by the cognitive state evaluation apparatus 100.

Thereafter, the cognitive state evaluation apparatus 100 performs modeling of learning-based personalized cognitive model (S1011), configures a customized task performance model using the personalized cognitive model (S1013) and obtains a substitution performance result of the task selected for each cognitive evaluation item using the customized task performance model (S1015).

And, the cognitive state evaluation apparatus 100 configures an analysis result interface for each cognitive evaluation item using the substitution performance result data (S1017), and provides the analysis result interface for each cognitive evaluation item to the guardian terminal 400 (S1019).

Thereafter, the guardian terminal 400 can output an analysis result report (S1021), and the cognitive state diagnosis apparatus 100 can provide cognitive enhancement track recommendation information based on the analysis result to the guardian terminal 400 (S1023).

FIGS. 6 to 7 are diagrams for exemplarily explaining a report interface outputted from the guardian terminal according to an embodiment of the present invention.

First, referring to FIG. 6, the analysis result interface can digitize and output the cognitive state which is based on the user's result data, and enhancement games for reinforcing insufficient elements and information on nearby hospitals can be outputted as additional information.

Also, referring to FIG. 7, the evaluation interface can process and output the user's result data as a pentagonal graph which is based on reference variables and can track, predict and output the changes over a period of time (two weeks). Also, since it is possible to identify the numerical value, etc. which have been changed by the reinforcement game, a function allowing the user to easily monitor and supplement his or her cognitive state can be provided.

The method according to the present invention described above may be implemented by a program which is executable on a computer and which is stored in a computer-readable recording medium. Examples of the computer-readable recording medium include ROM, RAM, CD-ROM, magnetic tapes, floppy disks, and optical data storage devices.

The computer-readable recording medium is distributed to a computer system connected through a network so that computer-readable codes can be stored therein and executed in a distributed manner. And, functional programs, codes and code segments for implementing the method can be easily inferred by programmers in the technical field to which the present invention pertains.

In addition, although preferred embodiments of the present invention have been shown and described above, the present invention is not limited to the specific embodiments described above, and various modifications can be made by those skilled in the technical field to which the invention pertains without departing from the gist of the present invention as claimed in the claims and these modifications should not be understood individually from the technical idea or perspective of the present invention.

1. A method of operating a cognitive state evaluation apparatus, comprising the steps of:
extracting first game data for cognitive evaluation from a user's response input information to a cognitive game application;
creating a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and
obtaining a substitution performance result of the cognitive task selected for each cognitive evaluation item using the customized cognitive task performance model and evaluating the cognitive ability of the user for each cognitive item based on the substitution performance result.

2. The method of operating a cognitive state evaluation apparatus according to claim 1, wherein the customized cognitive task performance model includes a virtual task performance model for predicting at least one of the user's execution time, error rate, and correct answer rate corresponding to the selected cognitive task and for outputting the substitution performance result.

3. The method of operating a cognitive state evaluation apparatus according to claim 1, wherein the cognitive architecture-based cognitive model includes an adaptive control of thought rational (ACT-R) architecture-based cognitive model, and the cognitive evaluation items include an attention deficit hyperactivity disorder (ADHD) evaluation items corresponding to the ACT-R model.

4. The method of operating a cognitive state evaluation apparatus according to claim 3, wherein the artificial intelligence learning-based user-customized cognitive model is configured by performing group classification associative learning to classify the feature information, which have been obtained by converting the game learning data for each cognitive task into a support vector machine (SVM), into an ADHD target group and a general subject group, and wherein the cognitive ability evaluation step includes calculating an ADHD item evaluation score of the user based on a classification model configured by the group classification association learning.

5. The method of operating a cognitive state evaluation apparatus according to claim 1, further comprising an accuracy verification step of further receiving additional game data different from the first game data from the user terminal from the user terminal and performing accuracy verification of the customized cognitive task performance model.

6. The method of operating a cognitive state evaluation apparatus according to claim 1, wherein the cognitive ability evaluation step comprises a result data processing step of configuring the result data according to the cognitive ability evaluation into an analysis interface and outputting it to a pre-registered guardian terminal corresponding to the user.

7. The method of operating a cognitive state evaluation apparatus according to claim 1, wherein the cognitive ability evaluation step includes further comprising a cognitive enhancement track recommendation step of recommending an enhancement task corresponding to a cognitive ability item evaluated below a preset threshold based on the result data.

8. The method of operating a cognitive state evaluation apparatus according to claim 1, wherein the cognitive game application includes a plurality of game interface applications configured step by step, corresponding to a task variable model for each pre-set cognitive evaluation item, and wherein the task variable model for each cognitive evaluation item includes at least one of a working memory variable model, an inhibition variable model, a divided attention variable model, a flexibility variable model, a processing speed variable model, and a selective attention variable model.

9. A cognitive state evaluation apparatus, comprising:
a game data processing unit for extracting first game data for cognitive evaluation from a user's response input information to a cognitive game application;
a customized cognitive task performance model configuration unit for creating a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and
a cognitive ability evaluation unit for obtaining a substitution performance result of the cognitive task selected for each cognitive evaluation item using the customized cognitive task performance model and evaluating the cognitive ability of the user for each cognitive item based on the substitution performance result.

10. A method of operating a user terminal device comprising the steps:
performing a cognitive game application corresponding to a preset cognitive diagnosis task for each cognitive evaluation item;
extracting first game data for cognitive evaluation from response input information for the cognitive game application; and
transmitting the first game data to a cognitive state evaluation apparatus, wherein
the cognitive state evaluation apparatus is configured to create a customized cognitive task performance model corresponding to the user by applying the extracted first game data to a user-customized cognitive model based on artificial intelligence learning that a cognitive architecture-based cognitive model has been pre-associatively trained in response to game data for each cognitive task; and obtain a substitution performance result of the cognitive task selected for each cognitive evaluation item using the customized cognitive task performance model and evaluating the cognitive ability of the user for each cognitive item based on the substitution performance result.

11. The method of operating a user terminal device according to claim 10, wherein the cognitive game application includes a plurality of game interface applications configured in stages, corresponding to a task variable model for each pre-set cognitive evaluation item, and wherein the task variable model for each cognitive evaluation item includes at least one of a working memory variable model, an inhibition variable model, a divided attention variable model, a flexibility variable model, a processing speed variable model and a selective attention variable model.

12. The method of operating a user terminal device according to claim 10, wherein
the user's response input information corresponding to the working memory variable model is information for inputting a series of sequentially displayed numbers in reverse order;
the user's response input information corresponding to the restraint variable model is object selection information corresponding to a stroop test query;
the user's response input information corresponding to the divided attention variable model is sequential number input selection information having different colors;
the user's response input information corresponding to the flexible variable model is card selection information suitable for a card classification criterion suggestion word; and
the user's response input information to the processing speed variable model is object selection information for selecting a figure having a different shape or a presented object.

13. The method of operating a user terminal device according to claim 12, wherein the task variable model for each cognitive evaluation item is determined according to the correct answer score and correct answer time information calculated from the user's response input information.

14-18. (canceled)
